(19) United States
(12) Patent Application Publication
(10) Pub. No.: US 2020/0054433 A1
(43) Pub. Date: Feb. 20, 2020

(54) EMBOLIC PROTECTION SYSTEMS AND DEVICES

(71) Applicant: MOKITA MEDICAL GMBH, Berlin (DE)

(72) Inventor: Richard Gerard VECCHIOTTI, Berlin (DE)

(73) Assignee: MOKITA MEDICAL GMBH, Berlin (DE)

(21) Appl. No.: 16/539,287

(22) Filed: Aug. 13, 2019

(30) Foreign Application Priority Data
Aug. 14, 2018 (GB) GB1813264.7

(51) Int. Cl. A61F 2/01; A61M 5/36; A61M 1/00

(52) U.S. Cl. CPC A61F 2/013; A61M 5/36; A61M 1/0062; A61M 2025/0042; A61M 2202/0486; A61M 2202/08; A61M 1/007

(57) ABSTRACT

A vascular catheter including an embolic protection system which is deployable within a blood vessel, comprising a filter arranged to block the flow of solid particles and a gas capture system for capturing gas bubbles entrained within a flow of blood along the blood vessel.

### BACKGROUND

Cerebral embolic protection devices are an emerging class of medical devices that are used during endovascular and percutaneous interventions. Procedures that intervene with vasculature that is in direct communication with the cerebral vasculature can put patients at risk of cerebral injury.

The clinical community generally believes that cerebral injuries arising from these interventions are caused by emboli travelling from the intervention site into the cerebral vasculature and obstructing blood flow through the process of embolization. The obstruction of blood flow disrupts, among other things, the oxygen supply to the brain, which results in damage to, and the subsequent death of, cells in the affected brain tissue.

Emboli have traditionally been assumed to consist of solid matter and debris, which are, predominantly, mechanically dislodged from the patient's anatomy during the intervention. Other sources of solid matter debris include parts and particles of the catheter-assembly itself that can be released, such as hydrophilic coating. Accordingly, several companies have developed cerebral protection devices designed to deflect, filter out, and optionally capture solid embolic debris to prevent it from reaching the cerebral vasculature. Examples of such devices include those disclosed in patent documents US 2011/0282379 A1 and US 2016/0324621 A1.

### SUMMARY OF THE INVENTION

According to one aspect of the invention, there is provided a vascular catheter including an embolic protection system which is deployable within a blood vessel, comprising: a filter arranged to block the flow of solid particles and a gas capture system for capturing gas bubbles entrained within a flow of blood along the blood vessel.

Emerging data from transcranial Doppler monitoring has established the prevalence of gaseous volumes entering the cerebral vasculature during endovascular and percutaneous interventions, and these gaseous volumes have been significantly correlated with the emergence of cerebral injury following such interventions.

There is a growing acceptance of the risks associated with air bubbles introduced during endovascular and percutaneous interventions, and there is a need for devices that protect against these risks.

The terms filter and mesh may be used interchangeably by those skilled in the art to refer to a device that allows blood to flow through but obstructs the flow of solids that are suspended in the blood. The gas capture system may draw in, or aspirate, gas bubbles through one or more ports for example. The filter blocks solid particles from flowing into the cerebral vascular.

In a preferred embodiment, the gas capture system comprises: an outer lumen comprising one or more openings; an inner lumen comprising one or more fluid collection ports; and a first source of pressure coupled to the inner lumen; wherein the inner lumen and the outer lumen are controllable to move relative to each other to bring at least one of the one or more openings of the outer lumen into alignment with at least one of the one or more fluid collection ports, and wherein the first source of pressure is controllable to draw gas bubbles into the one or more fluid collection ports.

The openings and fluid collection ports can be gaps, slots, holes or any other geometry that allows for fluid communication. The relative movement of the inner and outer lumina may be translational movement or rotational movement for example, or combinations thereof. When at least one fluid collection port is aligned with at least one opening, the gas capture system is said to be in an open configuration. Otherwise, it is said to be in a closed configuration, with the inner lumen fully isolated from fluid communication with surrounding fluid. Opening the gas capture system requires bringing at least one port into alignment with at least one opening, and closing the gas capture system requires taking all openings and ports out of alignment. The inner lumen is positioned within the outer lumen.

Drawing gas bubbles may also be referred to as aspirating.

To ensure there is no fluid communication in the closed position, an outer diameter of the inner lumen should be substantially the same as an inner diameter of the outer lumen, such that there is a tight fit between the inner lumen and outer lumen and no fluid can flow between them.

The first source of pressure may be connected directly to the inner lumen, for example to a first end of the inner lumen or through a side port, or it may be indirectly coupled to the inner lumen, for example through the outer lumen.

Preferably, the first source of pressure is a first syringe connected to a first syringe controller, a pressure source and pressure regulator, or a pump and pump controller.

Preferably, the first source of pressure is controllable to flush the inner lumen with a flushing fluid, preferably wherein the flushing fluid is one selected from the group comprising saline, glucose solution, dextrose solution, electrolyte solutions, fatty emulsions, and perfluorocarbons.

This allows the inner lumen to be flushed either in situ or prior to being positioned within the body. Flushing is ideally performed with the gas capture system in the closed configuration. The gas capture system can also be used to infuse saline by controlling the source of pressure to be positive relative to the blood pressure when the gas capture system is in the open configuration.

Optionally, the gas capture system may further comprise a second source of pressure coupled to the inner lumen.

The second source of pressure may be connected directly to the inner lumen, for example to a second end of the inner lumen or through a side port, or it may be indirectly coupled to the inner lumen, for example through the outer lumen.

Preferably, the second source of pressure is a second syringe connected to a second syringe controller, a pressure source and pressure regulator, or a pump and pump controller.

Optionally, the second source of pressure is controllable to flush the inner lumen with a flushing fluid, preferably wherein the flushing fluid is one selected from the group comprising saline, glucose solution, dextrose solution, electrolyte solutions, fatty emulsions, and perfluorocarbons.

Optionally, the inner lumen may be a catheter, microcatheter, guide catheter or specialised guidewire that provides the functions of a normal guidewire, but with a hollow channel to allow for the passage of fluid.

Using a guide catheter or specialised guide wire removes the need for a separate guidewire and reduces the size of the system.

Optionally, the gas capture system may comprise a sealing member at a distal end of the outer lumen. Preferably, the sealing member is one selected from a group comprising a duckbill valve, an elastomeric self-sealing valve and a combination duckbill and elastomeric self-sealing valve. A sealing member can be used to accommodate the passage of a guidewire through the inner lumen or outer lumen but prevents blood being drawn through the distal end of the lumen.

The proximal end of the vascular catheter or any other component, such as the lumina, is the end that is not inserted into the patient's body, and the distal end is the end that is inserted into the patient's body.

Optionally, the relative movement between the inner lumen and the outer lumen comprises translational movement. Alternatively or additionally, the relative movement between the inner and outer lumina may comprise rotational movement.

Preferably, the vascular catheter further comprises a control system positioned at a proximal end of the vascular catheter to control the relative movement of the inner and outer lumina.

Alternatively, the gas capture system comprises: an outer lumen comprising one or more openings; and a guidewire; wherein in use, the guidewire is controllable to move relative to the outer lumen to occlude one or more of the openings, and wherein gas bubbles are drawn into the openings as the guidewire is retracted relative to the outer lumen.

The guidewire is positioned within the outer lumen. The openings can be gaps, slots, holes or any other geometry that allows for fluid communication. When the guidewire is not occluding or blocking one or more of the openings, the gas capture system is said to be in an open configuration. Otherwise, it is said to be in a closed configuration, with the interior of the outer lumen fully isolated from fluid communication with surrounding fluid. Opening the gas capture system requires positioning the guidewire with a least one opening unblocked, and closing the gas capture system requires positioning the guidewire with all of the openings occluded.

To ensure there is no fluid communication in the closed position, an outer diameter of the guidewire should be substantially the same as an inner diameter of the outer lumen, such that there is a tight fit between the guidewire and outer lumen and no fluid can flow between them.

As the guidewire is retracted past the openings, it creates a lower-pressure zone within the outer lumen that draws fluid into the outer lumen through the openings.

Optionally, the gas capture system may comprise a sealing member at a distal end of the outer lumen. Preferably, the sealing member is one selected from a group comprising a duckbill valve, an elastomeric self-sealing valve and a combination duckbill and elastomeric self-sealing valve. A sealing member allows the passage of the guidewire through the outer lumen but prevents blood being drawn through the distal end of the outer lumen.

Preferably, the gas capture system is positioned at a location where gas bubbles accumulate. Optionally, the gas capture system may be positioned at a leading edge of the filter. Alternatively or additionally, the gas capture system may be positioned at an apex of the filter.

Positioning the gas capture system at a location where gas bubbles are known or anticipated to accumulate when and if introduced into the vascular system, such as a leading edge or apex of the filter, allows for improved gas capture. This location may be a particular region of the vascular system or may be on a component of the vascular catheter or embolic protection system for example. Ideally the openings and fluid collection ports will be centrally positioned in such a location.

Preferably, the embolic protection system further comprises a bubble capture area. Optionally, this bubble capture area may be a detent. Having such a bubble capture area means that gas bubbles collect in a known location, and the gas capture system can be positioned in this region to improve gas capture.

Optionally, the filter may be arranged to trap solids. Alternatively, the filter may be arranged to deflect solids.

When arranged to deflect rather than capture solids, the filter blocks solid particles from flowing into the cerebral vascular but does not remove them from the blood flow, instead allowing them to flow down the aorta away from the cerebral vasculature.

According to another aspect of the invention, there is provided an embolic protection system comprising a catheter having a deployable filter arranged to block the flow of solid particles and a gas capture system for capturing gas bubbles entrained within a flow of blood along a blood vessel.

According to another aspect of the invention, there is provided a gas capture system comprising: an outer lumen comprising one or more openings; and, a guidewire; wherein in use, the guidewire is controllable to move relative to the outer lumen to occlude one or more of the openings, and wherein gas bubbles are drawn into the openings when the guidewire is retracted relative to the outer lumen.

The guidewire is positioned within the outer lumen. The openings can be gaps, slots, holes or any other geometry that allows for fluid communication. When the guidewire is not occluding or blocking one or more of the openings, the gas capture system is said to be in an open configuration. Otherwise, it is said to be in a closed configuration, with the interior of the outer lumen fully isolated from fluid communication with surrounding fluid. Opening the gas capture system requires positioning the guidewire with a least one opening unblocked, and closing the gas capture system requires positioning the guidewire with all of the openings occluded.

To ensure there is no fluid communication in the closed position, an outer diameter of the guidewire should be substantially the same as an inner diameter of the outer lumen, such that there is a tight fit between the guidewire and outer lumen and no fluid can flow between them.

As the guidewire is retracted past the openings, it creates a lower-pressure zone within the outer lumen that draws fluid into the outer lumen through the openings.

According to a further aspect of the invention, there is provided a method of providing embolic protection during a vascular procedure, comprising positioning a catheter within a blood vessel and controlling a gas capture system to capture gas bubbles introduced during the procedure and entrained within a flow of blood along the blood vessel.

Gas bubbles may be introduced by being released from devices used during the procedure for example, or they may be introduced through other means such as open vascular access.

In this method, the catheter comprises the gas capture system.

Preferably, controlling a gas capture system to capture gas bubbles comprises aspirating gas bubbles through one or more openings. This allows the bubbles to be removed from the vascular system.

Preferably, the gas bubbles are captured from a predetermined location at which the gas bubbles accumulate. Positioning the gas capture system at a location where gas bubbles are known or anticipated to accumulate when and if introduced into the vascular system allows for improved gas capture.

### BRIEF DESCRIPTION OF DRAWINGS

Examples of the present invention will now be described in detail with reference to the accompanying drawings, in which:

FIG. 1 shows a vascular catheter:

FIGS. 2a and 2b show a gas capture system comprising an inner lumen and an outer lumen;

FIGS. 3a and 3b show alternative configurations of a gas capture system;

FIGS. 4a and 4b show an embolic protection system arranged to deflect solid particles and capture gas bubbles;

FIGS. 5a and 5b show an alternative configuration of an embolic protection system arranged to deflect solid particles and capture gas bubbles;

FIGS. 6a-c show a control system for controlling the position of the lumina of the gas capture system of FIG. 2a;

FIG. 6d shows two syringes for controlling the flow of fluid through the inner lumen of the gas capture system of FIG. 2b;

FIG. 7 shows an embolic protection system arranged to capture solid particles;

FIG. 8a shows the gas capture system of FIG. 2a integrated into the embolic protection system of FIG. 7;

FIGS. 8b and 8c show close-up views of the gas capture system of FIG. 8a;

FIGS. 9a, 9c and 9e show a control system for controlling the position of the lumina of the gas capture system of FIG. 2a; and,

FIGS. 9b and 9d show the respective positions the lumina when the control systems are configured as shown in FIGS. 9c and 9e;

FIGS. 10a and 10b show cutaway views of gas capture system without an inner lumen;

FIG. 10c shows the proximal end of the gas capture system of FIGS. 10a and 10b; and,

FIG. 11 shows a flowchart of a method for capturing gas bubbles.

### DETAILED DESCRIPTION

The present invention provides an improved embolic protection system for use during endovascular and percutaneous interventions, in particular for capturing gas bubbles that may enter the cerebral vasculature during such interventions. The embolic protection system is deployed in situ within a blood vessel using a catheter.

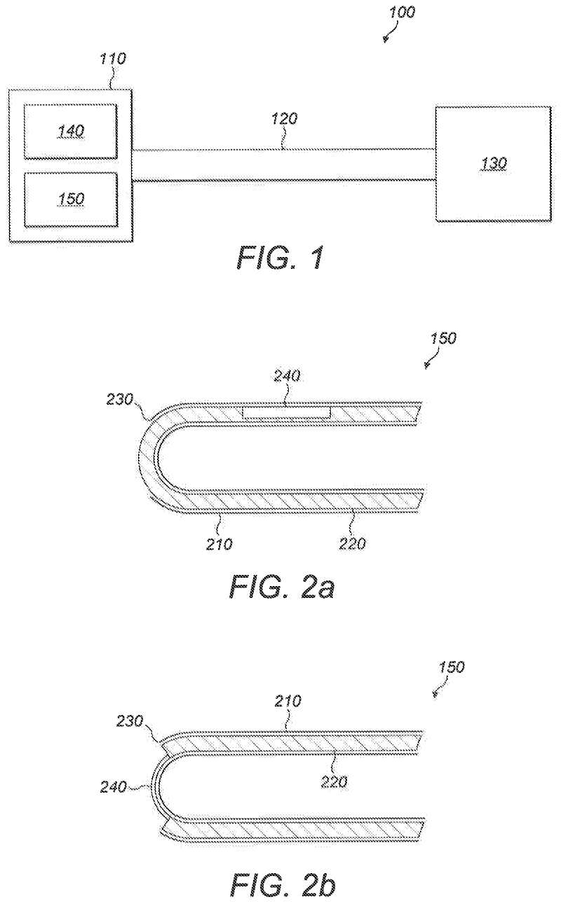

An embolic protection system is shown in FIG. 1. A catheter 100 comprises a tubular sheath 120, with an embolic protection system 110 at a distal end of the catheter 100, and a control system 130 at a proximal end of the catheter 100. The control system 130 may be used to control the position of the catheter 100 within a patient's body, as well as to deploy, position and control the embolic protection system 110.

The purpose of the embolic protection system 110 is to mitigate or prevent solid particles and gas bubbles in the blood flowing to the cerebral vasculature. To achieve this, the embolic protection system 110 comprises both a filter 140 arranged to block the flow of solid particles to the cerebral vasculature or remove solid particles from the vasculature, and a gas capture system 150 arranged to capture gas bubbles. The embolic protection system 110 is generally deployed in a patient's aortic arch and/or precerebral vessels to reduce the quantity of solid particles and gas bubbles that flow into one or more of the brachiocephalic artery, right subclavian artery, right common carotid artery, left common carotid artery and left subclavian artery. Other areas of the anatomy in which the embolic protection system 110 may potentially be deployed include the left ventricle, the aortic root and the ascending aorta.

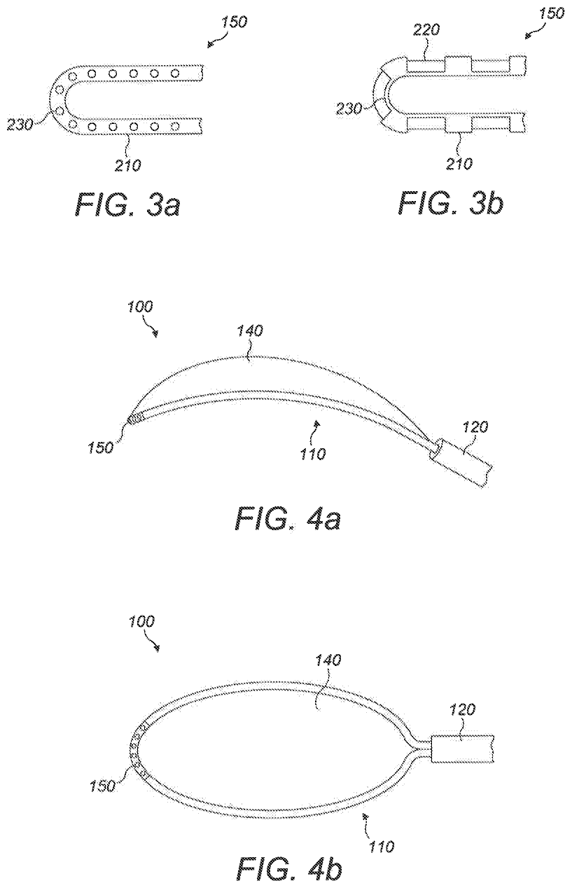

An embodiment of the gas capture system 150, shown in FIGS. 2a and 2b, comprises an outer lumen 210 and an inner lumen 220. The lumina 210 and 220 are shown with a u-shaped bend, but other arrangements, such as straight, are also envisaged; the actual arrangement will depend on the particular application. The inner lumen 220 is shaped to fit closely within the outer lumen 210 but loosely enough to allow the inner lumen 220 to move slidably within the outer lumen 220, i.e. the outer diameter of the inner lumen is approximately the same as the inner diameter of the outer lumen.

The lumina 210 and 220 comprise respective openings 230 and 240. To differentiate between the openings of the two lumina, the opening 240 on the inner lumen 220 is referred to as a fluid collection port 240. The terms opening, port, orifice, slot and window may be used interchangeably to refer to an opening or gap in the wall of a lumen.

In use, the gas capture system 150 is generally immersed within a patient's blood flow. In FIG. 2a, the opening 230 and the port 240 are not aligned, such that the gas capture system is in a closed configuration with the interior of the inner lumen 220 fully isolated from intravascular communication. The open configuration of the gas capture system 150 is shown in FIG. 2b, wherein the opening 230 and port 240 are aligned such that there is communication between the interior passage of the inner lumen 220 and the surrounding blood flow.

Transition between the closed and open configurations in FIGS. 2a and 2b is achieved by sliding the inner lumen 220 axially within the outer lumen 210 to bring the opening 230 and port 240 into or out of alignment. Alternative arrangements where the inner lumen 220 rotates relative to the outer lumen 210 to bring the port 240 and opening 230 into alignment are also envisaged.

In use, the inner lumen 220 is filled with sterile saline or any other fluid suitable for medical use. As described in more detail below, at least a first end of the inner lumen 220 is coupled to a first source of pressure, and a second end of the inner lumen 220 may either be open, closed, or coupled to a second source of pressure. When the gas capture system 150 is in the closed configuration, the fluid within the inner lumen 220 flows in isolation from the intravascular blood flow, controlled by the first and/or second sources of pressure.

Alternatively, a second source of pressure may be connected to a second end of the outer lumen 210 in the case that the inner lumen 220 does not run the full length of the outer lumen 210. In this configuration, the second source of pressure is indirectly coupled to the inner lumen 220.

To capture gas bubbles, the first and/or second sources of pressure are controlled to create a lower pressure within the inner lumen 220 relative to the intravascular pressure, with the gas capture system 150 in the closed configuration. The inner lumen 220 is then moved relative to the outer lumen 210 until the opening 230 and port 240 are at least partially aligned, i.e. the open configuration. The pressure gradient then causes the surrounding gas bubbles, and some of the surrounding blood, to be drawn, or aspirated, into the inner lumen 220 via the fluid collection port 240.

Throughout this disclosure, the use of the terms negative pressure or lower pressure should be understood to mean a pressure that is lower than the intravascular blood pressure unless otherwise indicated.

Blood that is collected can be filtered from debris and air prior to being circulated back into the patient via the same vascular entry or another vascular entry (for example femoral venous return) if desired.

These steps can be reversed, i.e. a lower pressure can be applied after opening the gas capture system 150.

The same method can be used for intravascular sampling, or fluid can be infused by using a source of positive pressure.

Although the gas capture system in FIGS. 2a and 2b has a single opening 230 and a single fluid collection port 240 of a similar size to the opening 230, alternative configurations are envisaged in which there are additional openings or ports, and/or in which the openings and ports are of different shapes and sizes. For example, the inner lumen 220 may instead comprise one large opening which can be brought into alignment with one or more of a plurality of openings on the outer lumen 210, or this arrangement may be reversed with the outer lumen 210 having one large opening 230 and the inner lumen 220 having a plurality of smaller ports 240. The outer lumen 210 may alternatively comprise a gap along its trajectory, i.e. an opening that extends around the entire circumference of the outer lumen 210.

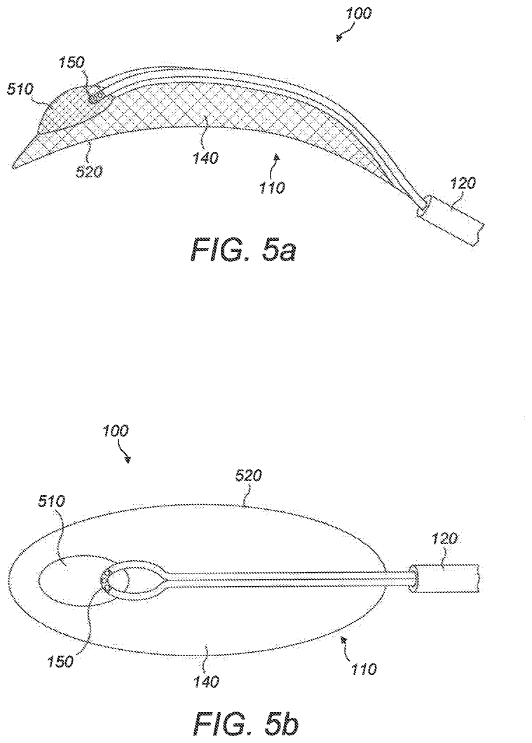

Examples of such alternative configurations are shown in FIG. 3a, in which the gas capture system 150 comprises an outer lumen 210 with a plurality of circular openings 230, and in FIG. 3b, in which the gas capture system 150 comprises an outer lumen 210 with a plurality of elongated rectangular openings 230.

### First Embodiment

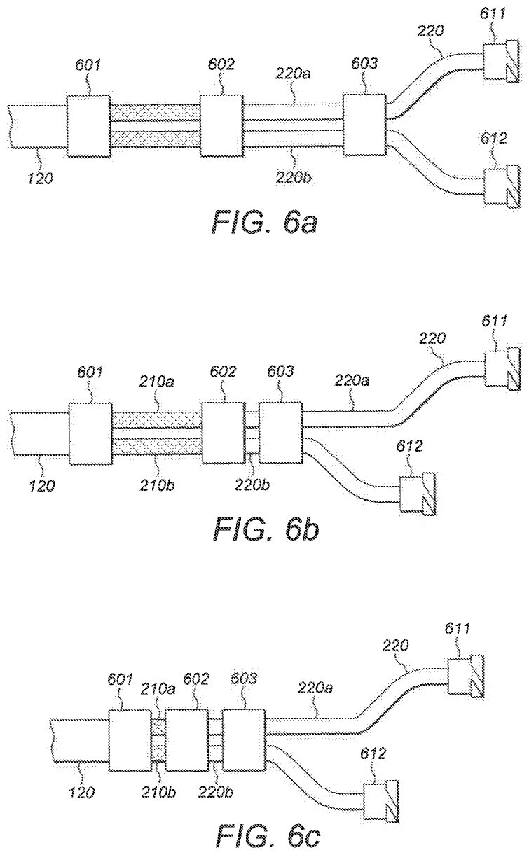

The gas capture system 150 described above is designed for use in combination with a filter arranged to block the flow of solid particles. FIGS. 4a and 4b show such an embodiment of a vascular catheter 100, in which the embolic protection system 110 comprises the above gas capture system 150 and a filter 140 arranged to deflect solids. The filter 140 may be constructed of metallic or polymeric materials for example, or any other material suitable for use as a medical-grade filter.

In use, the deflecting filter 140 is positioned within a patient's aortic arch covering one or more of the brachiocephalic artery, left common carotid artery and left subclavian artery. The filter 140 blocks solid particles above a certain size from flowing into these arteries and deflects them down the aorta away from the cerebral vascular, where they are eventually pumped to the lungs or trapped in peripheral regions of the vascular system, where they are less harmful.

Rather than being deflected, at least some gas bubbles entrained in the patient's blood flow gather on the deflecting filter 140. Certain parts of the filter 140, such as the edges or the apexes, act as preferred nucleation sites where gas bubbles are more likely to accumulate. In the embodiment illustrated in FIGS. 4a and 4b, the gas capture system 150 is positioned at the leading edge of the filter 140 to maximise the potential for gas capture, although alternative embodiments are envisaged in which the gas capture system 150 is positioned at different parts of the filter, particularly at the peripheral edges.

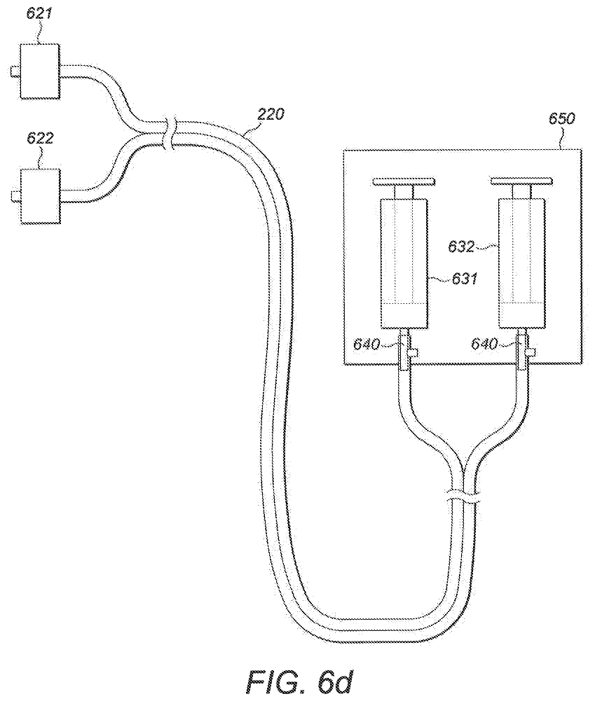

FIGS. 5a and 5b show a similar embolic protection system 110, again comprising a defecting filter 140 and a gas capture system 150, but with the additionally comprising a detent 510 designed to act as a preferred nucleation site for bubbles to collect. The gas capture system 150 is positioned in the detent 510 in order to increase gas capture. Alternative configurations are envisaged with the detent zone in a different position, or with multiple detent zones. The embodiments in FIGS. 4a and 4b and 5a and 5b could also be combined, i.e. a gas capture systems 150 could be positioned at both the leading edge of the filter 140 and in a detent region 510.

The edges of the filters 140 in FIGS. 4a-b and 5a-b are provided with a frame comprising a pseudoelastic shape-memory material, such as nitinol, which allows the embolic protection system 110 to return to its original shape after being compressed or folded, such as within the sheath 120 prior to deployment. For example, the frame could be the outer lumen 210, or could be a pseudoelastic shape-memory element, such as a wire, running adjacent to the outer lumen 210, or a pseudoelastic shape-memory element, such as a wire, within the outer lumen 210 or inner lumen 220. Such a frame 520 is visible in FIGS. 5a and 5b.

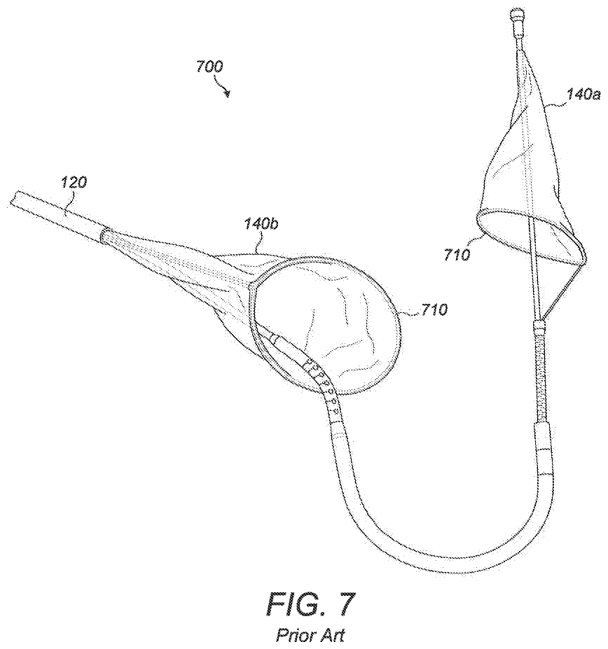

Moving on to FIGS. 6a-d, components of a control system 130 for controlling the pressure within the inner lumen 220 and the movement of the outer lumen 210 and inner lumen 220 are shown in FIGS. 6d and 6a-c respectively. The illustrated control system components are for an embodiment in which both the ends of the inner lumen 220 are coupled to sources of pressure, such as the embodiments in FIGS. 4a-b and 5a-b.

To adjust the relative positions of the outer lumen 210 and inner lumen 220, and thereby transition the gas capture system 150 between the open and closed configurations, the control system 130 comprises three base stages, 601-603. Base stage 601 is fixed to the sheath 120, base stage 602 is fixed to both ends of the outer lumen 210, and base stage 603 is fixed to one leg of the inner lumen 220, in this case the lower leg, and free to slide over the other leg of the inner lumen 220, in this case the upper leg.

FIG. 6a shows the arrangement of the base stages 601-603 when the gas capture system is in the closed position. To transition to the open position, base stage 603 is moved towards base stage 602 into the configuration shown in FIG. 6b. Base stage 603 is fixed to the lower leg of the inner lumen 220, so this relative movement causes section 220b of the inner lumen 220 to slide into section 210b of the outer lumen 210. The inner lumen 220 is continuous, so this process also causes a corresponding length of the inner lumen 220 to slide out of section 210a of the outer lumen 210. Base stage 603 slides along section 220a of inner lumen 220 during this process.

The relative movement of the outer lumen 210 and inner lumen 220 causes the openings 230 and fluid collection ports 240 to become aligned in the open configuration of the gas capture system 150. The amount by which the base stage 603 must be moved relative to base stage 602 depends on the relative movement required to align the openings 230 and ports 240.

The gas capture system itself can also be advanced relative to the sheath 120 by moving base stage 602 towards base stage 601. Base stage 602 is fixed to both ends of the outer lumen 210, so moving it towards base stage 601, which is fixed to the sheath 120, causes sections 210a and 210b of the outer lumen 210 to slide into the sheath 120, thereby allowing the gas capture system to advance out of the distal end of the sheath 120. The relative positions of the outer lumen 210 and inner lumen 220 are unaffected by this transition, so the gas capture system 150 will remain in whichever of the open and closed configurations it was already in. Advancing the gas capture system 150 in this way allows for better access to the full bubble capture area.

The closed and open configurations could be reversed depending on the particular arrangement of the openings 230 and ports 240, i.e. moving base stage 603 towards base stage 602 could instead transition the gas capture system 150 from the open to the closed configuration.

In FIGS. 6a-c, the inner lumen 210 comprises female luer fittings 611 and 612 at each end, allowing for connection to the male luer fittings 621 and 622 shown in FIG. 6d. These fittings allow for easy attachment of pressure sources, but they are optional components and it should be understood that such fittings are intended to represent a continuation, rather than termination, of the inner lumen 220.

The control system 130 further comprises first and second pressure sources in the form of first and second syringes 631 and 632 respectively, each connected to an end of the inner lumen 220 using a three-way luer valve 640. The syringes 631 and 632 are controlled by a syringe controller 650 which can independently manipulate the syringes 631 and 632 to apply a positive or negative pressure gradient with respect to the intravascular pressure. The syringes 631 and 632 can be filled or emptied independently with saline or other fluids as needed by use of the three-way luer valves 640. Although the illustrated embodiment shows a pair of syringes controlled by a syringe controller, these pressure sources could be replaced with any suitable controllable pressure source.

Prior to deployment, the embolic protection system 110 is compressed in a retracted position within the sheath 120, with the gas capture system 150 arranged in the closed configuration, i.e. with the openings 230 and fluid collection ports 240 not aligned. Either prior to deployment, or during deployment, the inner lumen 220 is flushed with saline or a similar flushing fluid such as perfluorocarbon, preferably by pumping the saline from one end of the inner lumen 220 until all air is displaced.

The embolic protection system 110 is then deployed by inserting the distal end of the catheter 100 into the vascular system, for example through a surgical incision, and guiding it into place in the aorta, at any point between left ventricle and precerebral vessels, possibly along a guidewire running through the sheath 120 adjacent to the lumina. The embolic protection system 110 is then ejected from the sheath 120 and arranged to cover one or more of the brachiocephalic artery, left common carotid artery and left subclavian artery. The filter 140 expands from its compressed configuration within the sheath 120 to its illustrated uncompressed configuration due to the pseudoelastic frame.

Once all air is displaced and the embolic protection system 110 is ready to be positioned or is already in position within the vasculature, the two ends of the inner lumen 220 are connected to the syringes 631 and 632 respectively. Gas bubbles can then be captured by lowering the pressure within the capture system relative to the intravascular pressure using one or both of the syringes 631 and 632 and moving the base stage 603 towards base stage 602 to open the fluid collection ports 240 and draw in gas bubbles. During gas capture, the gas capture system 150 can be advanced further out of the sheath 120 by moving base stage 602 towards base stage 601, thereby affording greater positional control and better access to the full bubble capture area.

At the end of the intervention, the fluid collection ports are closed by moving base stage 603 away from base stage 602. If necessary, one of the syringes 631 or 632 can be used to apply a positive pressure to flush the inner lumen 220 in order to remove captured gas bubbles and any blood captured during the gas capture process. The captured fluids are optionally flushed into a drain, where they can be processed to remove gas bubbles and then be reintroduced to the vasculature if desired.

The embolic protection system 110 can then be withdrawn into the sheath 120 and the catheter 100 can be removed from the vasculature.

### Second Embodiment

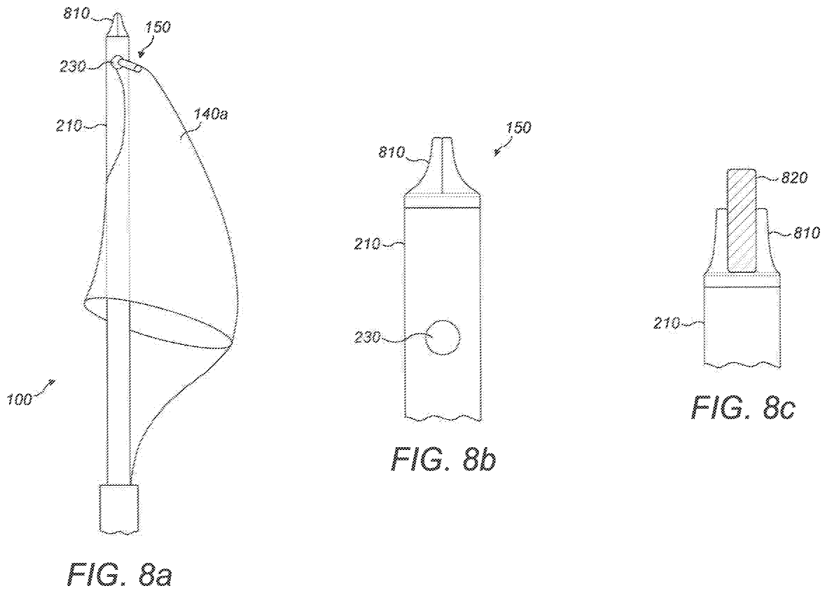

A known embolic protection system 700, different to that shown in FIGS. 4a-b and 5a-b, is shown in FIG. 7. Unlike the deflecting filter in FIGS. 4a-b and 5a-b, the filters 140a and 140b of the device 700 are substantially conical and arranged to filter and capture solid particles such that they are removed from the blood flow rather than deflected downstream. Although the illustrated example comprises two filters, 140a and 140b, the embolic protection system 700 may comprise additional or fewer filters depending upon the application. The embolic protection system 700 shown in FIG. 7 does not comprise a gas capture system.

Each filter 140a, 140b of the device 700 comprises a pseudoelastic shape-memory frame 710, for example made of nitinol, that causes the filter to return to an open configuration following deformation or compression, such as when compressed awaiting deployment from a catheter.

During an intervention, the embolic protection system 700 is inserted into the aortic arch via the brachiocephalic artery. The distal end of the device including filter 140a is directed into the left common carotid artery. Once in position, one filter 140b is deployed in the brachiocephalic artery, and the other filter 140a is deployed in the left common carotid artery, and the filters thereby capture solid particles that flow into these arteries. The filters 140a and 140b are later retracted into the sheath 120 at the end of the procedure, along with trapped solid particles, and the catheter 100 is removed. Although the illustrated device 700 is described protecting the brachiocephalic and left common carotid arteries, filters can be positioned any combination of precerebral vessels branching off the aortic arch as necessary.

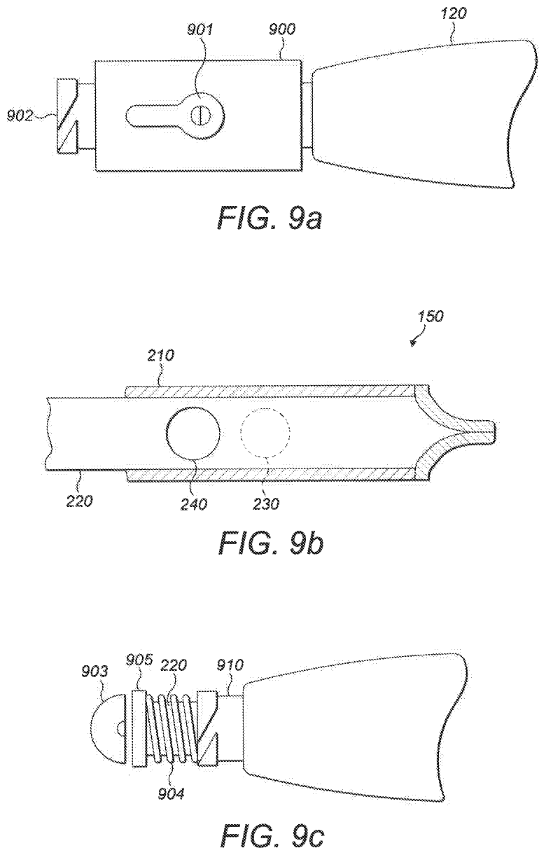

A catheter 100 according to an embodiment of the present invention, integrating the gas capture system 150 with the embolic protection system 700, is shown in FIG. 8a. The opening 230 in the outer lumen 210 is positioned near to the apex of the filter 140a, as this is an area where bubble nucleation is particularly likely. A similar configuration is possible for each filter 140a and 140b of the embolic protection system 700 allowing for gas capture at one or both of filters 140a and 140b, wherein a gas capture system 150 is positioned at an apex of the filter, although alternative embodiments are envisaged with openings at other positions, such as at the edges of the filter. During use, the gas capture system 150 may also draw small solid particles that collect at the apex of the filter in additional to gas bubbles.

The outer lumen 210 could have openings 230 located at the apex of one or both filters 140a and 140b. Similarly, the inner lumen 220 could have ports 240 located for use at the apex of one or both of filters 140a and 140b. In an alternative arrangement, a second inner lumen is utilized, within the first inner lumen 220, with additional ports to allow for independent gas capture control at each filter 140a, 140b not being managed by the first inner lumen 220 and its corresponding ports 240. Additionally, this smaller inner lumen residing inside lumen 220 could be a specialized catheter, microcatheter, guide catheter, or guidewire with ports built in to afford the same functionality as ports 240 of the inner lumen 220.

The arrangement in FIG. 8a comprises a sealing member in the form of a duckbill valve 810 at the distal end of the catheter 100, which allows for the passage of a guidewire 820 through the inner lumen, as shown in FIG. 8c. Unlike the first embodiment, in which the inner lumen formed a loop with both ends coupled to a source of pressure at the proximal end of the catheter 100, the outer lumen 210 and inner lumen 220 in FIGS. 8a-c terminate at the valve 810 at the distal end of the catheter 100. The valve 810 is connected to the end the outer lumen 210 in the illustrated embodiment, but alternative embodiments are envisaged in which the valve 810 is fixed to the inner lumen 220.

FIG. 8b shows a close-up view of the distal end of the catheter 100, in particular, the opening 230 in the outer lumen 210 and the duckbill valve 810. In use, the duckbill valve 810 prevents fluid being drawn through the end of the catheter when a lower pressure is applied to the inner lumen 220, while still allowing a guidewire 820 to travel along the inner lumen 220. The valve 810 prevents fluid being drawn both when the guidewire is present, by sealing around the guidewire 820, and when it is not. In alternative configurations, the inner lumen 220 and/or outer lumen 210 may terminate in a closed end without a sealing member, and a guidewire 820 may run alongside, rather than within, the lumina, or within the outer lumen 210 but outside the inner lumen 220.

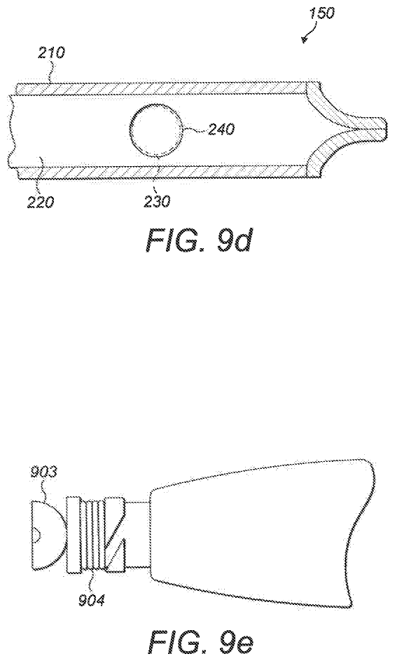

The mechanism for controlling the relative positions on the inner lumen 220 and outer lumen 210 according to the second embodiment, and the corresponding positions of the lumina, are shown in FIGS. 9a, 9c and 9e and FIGS. 9b and 9d respectively.

The control mechanism comprises an external lever 901 and a casing 900 that terminates in a luer fitting 902, as shown in FIG. 9a, to which a source of pressure, such as a syringe, is connected during use. The lever 901 is connected by a cam shaft to a cam 903 housed within the casing, shown in FIGS. 9c and 9e. When the lever 901 is rotated, the cam 903 rotates with it.

The casing 900 is connected to a luer fitting 910 which is fixed to an end of the outer lumen 210 (not visible in FIGS. 9a, 9c and 9e), such that the casing 900, and therefore the control mechanism as a whole, is fixed relative to the outer lumen 910.

The cam 903 abuts against a rim 905 of the inner lumen 220. When the cam 903 is in the disengaged position shown in FIG. 9c, the inner lumen 220 and outer lumen 210 are positioned in the closed configuration shown in FIG. 9b, with the opening 230 and fluid collection port 240 out of alignment. The opening 230 is overlaid onto the cross-sectional view in FIGS. 9b and 9d with a dashed line.

A spring 904 exerts a force between the luer fitting 910 connected to the outer lumen 210 and the lip 905 of the inner lumen to resist the inner lumen 220 moving into the outer lumen 210, thereby biasing the gas capture system 150 in the closed configuration unless intentionally opened.

When the cam 903 is rotated 180 degrees about its axis to the configuration shown in FIG. 9e, it exerts a force on the lip 905 of the inner lumen 220, compressing the spring 904 and causing the inner lumen 220 to slide distally into the outer lumen 210, i.e. the cam 903 converts the rotational motion of the lever 901 into an axial translation of the inner lumen 220. At the distal end of the catheter 100, this causes the gas collection system 150 to transition into the open configuration with the fluid collection port 240 aligned with the opening 230.

As with the first embodiment, the open and closed configurations of the control mechanism may be reversed depending on the particular arrangement of the openings 230 and ports 240.

Alternative configurations are envisaged in which the cam rotation can be limited by a ratchet mechanism to prevent counter rotation, or decoupling or self-destructing features that disengage the camshaft from the lever 901.

Further alternative configurations are envisaged in which the entire inner lumen 220 is replaced by a specialized catheter, microcatheter, guide catheter, or guidewire comprising fluid collection ports 240 at one or both filter sites 140a or 140b. In this configuration, the cam control mechanism and lever would be obviated and the position of the guidewire could instead be controlled manually at the proximal end of the catheter by way of translation mediated by visual aids. Such visual aids could include coloured bands on the guidewire to serve as a translation stop cue, for example to indicate the position at which the ports 240 and openings 230 are aligned. Such coloured bands could also be radiopaque to allow the operator to control translation under fluoroscopy. Alternatively, the specialized catheter, microcatheter, guide catheter, or guidewire could be coupled to the cam control mechanism described earlier.

Prior to deployment, the embolic protection system 110 is compressed in a retracted position within the sheath 120, with the gas capture system 150 arranged in the closed configuration, i.e. with the openings 230 and fluid collection ports 240 not aligned. The inner lumen 220 is flushed with saline, or a similar flushing fluid such as perfluorocarbon, to remove air, by firstly pumping the saline from the proximal end of the inner lumen 220 with the openings in the closed configuration, such that the air and flushing fluid are forced out through the sealing member 810. Optionally, the fluid collection ports 240 can be opened to allow for the flushing solution to be routed through the collection ports 240.

The first flushing stage is skipped in embodiments without a sealing member, and the catheter 100 is flushed only with the ports in the open configuration.

The gas capture system 150 is then closed and the embolic protection system 110 is deployed by inserting the distal end of the catheter 100 into the vascular system and directed to the aortic arch. The gas capture system 150 can also be deployed over a guidewire. If a guidewire is to be in place prior to the deployment of gas capture system 150, a peel-away sheath feature may be employed in some embodiments of the system to facilitate the loading of the duck-bill end of the device onto the guidewire. Separate filters are ejected from a compressed configuration within the sheath 120 and positioned in one or more of the brachiocephalic artery, right subclavian artery, right common carotid artery, left common carotid artery and left subclavian artery. The filters 140a and 140b expand from their compressed configuration due to the pseudoelasticity of the frames 710.

Once the embolic protection system 110 is in position, gas bubbles and small debris particles can be captured by applying a lower pressure using a syringe, or an alternative external pressure source, connected to the luer fitting 902 at the proximal end of the inner lumen 220, and then rotating the lever 901 to open the ports 240 of the gas capture system 150. If necessary, the guidewire can be removed prior to applying a lower pressure, for example if the guidewire is large enough to significantly obstruct the flow of fluid through the inner lumen 220.

At the end of the intervention, the fluid collection ports 240 are closed by rotating the lever 901 back to the closed position, and the application of the lower pressure is stopped. The captured fluids are optionally processed to remove gas bubbles and then reintroduced into the vasculature if desired. Blood collected can be filtered from debris and air prior to being circulated back into the patient via the same vascular entry or another vascular entry (for example femoral venous return).

The embolic protection system 110 can then be withdrawn into the sheath 120 and the catheter 100 can be removed from the patient's body.

### Third Embodiment

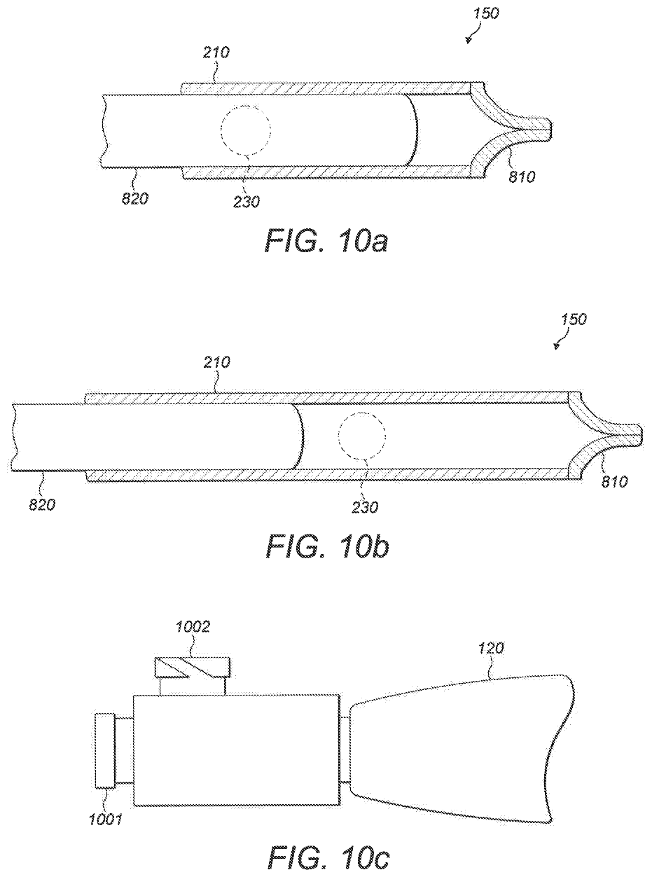

In an alternative embodiment, the gas capture system does not utilise an inner lumen. Instead, a close-fitting catheter, microcatheter, guide catheter, or guidewire with an outer diameter similar to that of the inner diameter of the outer lumen is used to open and close the openings of the outer lumen. Ideally, there is a loose interference fit between the guidewire and the outer lumen, such that fluid cannot flow in a space between the guidewire and the outer lumen but the guidewire can still be moved translationally relative to the outer lumen. This embodiment is similar to the second embodiment described above, and is designed to be integrated with the embolic protection system 700 in the same manner as described above with reference to the second embodiment, although it could also be used as a standalone gas capture system for embolic protection. This third embodiment is illustrated in FIGS. 10a-c. In FIG. 10a, the guidewire 820 is positioned within the outer lumen 210 blocking the opening 230, represented by a dashed line, such that the close fit within the outer lumen causes the opening to be closed. As with the second embodiment above, the outer lumen 100 comprises a duckbill valve 810 at its distal end, which allows for the passage of the guidewire 820 through the outer lumen 210. Although only one opening is shown, embodiments with more openings and openings of different shapes and sizes are envisaged, as with previous embodiments.

At the proximal end of the catheter 100, the guidewire travels through a substantially air-tight and liquid-tight seal in the form of a self-sealing, self-conforming, and pierceable entry port 1001, although other sealable entry port features may be employed such as manually sealable iris style ports.

The embolic protection system 110 is inserted into a patient over the guidewire 820, i.e. it is placed and deployed with the guidewire 820 in place, thus keeping the opening 230 sealed when the embolic protection system 110 enters the patient.

Once the embolic protection system 110 is positioned, deployed, and substantially anchored, the guidewire 820 can be retracted into the outer lumen 210. As the guidewire is retracted through the duck-bill valve 810, the valve seals itself and thereby prevents blood and other bodily fluids from entering through the distal end of the outer lumen 210.

As the guidewire 820 is retracted further past the opening 230, the gas capture system 150 transitions into the open configuration (i.e. fluid is able to flow from the vascular system into the outer lumen). The expansion of empty space created as the guidewire tip is pulled backward creates a low-pressure zone which draws gas bubbles, blood, other bodily fluids, and debris into the outer lumen 210. Completely withdrawing the guidewire 810 all the way out of the outer lumen 210 allows for a maximal volume of gas bubbles, blood, bodily fluid, and debris to be retained inside the outer lumen 210.

The captured volume of gas bubbles, blood, bodily fluid, and debris can be fully removed from the outer lumen 210 by way of a side port 1002 at the proximal end of the outer lumen 210, with removal accomplished for example by using a syringe or syringe mechanism attached to the side port or by connecting a light vacuum line to the side port to selectively remove the captured volume of gas bubbles, blood, and bodily fluid. A light vacuum line can also be applied continuously to ensure the directionality of the collection process and to ensure that captured gas bubbles, blood, bodily fluid, and debris are not reintroduced into the arterial bloodstream.

Once the captured fluid has been removed as necessary, the guidewire 820 can be reintroduced into the catheter through the entry port 1001, past the opening 230, thus closing it, and out of the valve 810. The embolic protection system 110 can then be retracted into the sheath 120 and the catheter 100 can then be removed from the vasculature by withdrawing it over the guidewire 820.

Alternatively, the guidewire 820 may not be reintroduced, and application of the light vacuum may be prolonged to prevent the reintroduction of gas bubbles, blood, bodily fluid, and debris while the embolic protection system is retracted and the catheter 100 is removed without the guidewire in place.

Any blood/bodily fluid collected can be filtered from gas and debris and then reintroduced into the patient via femoral venous return (or similar method of reintroduction) if desired.

Method

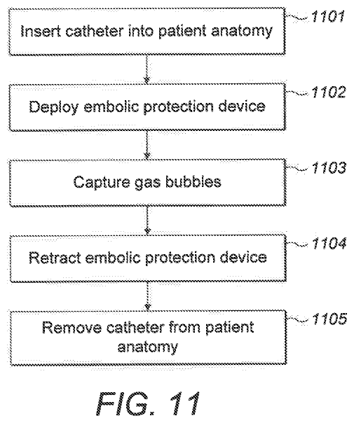

Exemplary method steps for capturing gas bubbles are shown in FIG. 11. At step 1101, the catheter 100 is inserted in to patient's anatomy, for example through a surgical incision, and guided into position. Once in position, the embolic protection system 110 is deployed at step 1102. Gas bubbles can be captured by using the gas capture system 150 of the embolic protection system at step 1103. Once all gas capture is complete, the embolic protection system 110 can be retracted into the sheath 120 at step 1104, before the catheter is removed from the patient's anatomy at step 1105.

Any blood/bodily fluid collected can be filtered from gas and debris and then reintroduced into the patient via femoral venous return (or similar method of reintroduction) if desired and any point during or after the capturing of gas bubbles is initiated.

In all embodiments described above, the order in which the openings and fluid collection ports 230 and 240 are opened and the lower pressure is applied can be reversed if necessary during the gas capture stage, or these can be performed simultaneously. A guidewire, if used, may be removed and reintroduced as necessary throughout the procedure.

The gas capture system 150 may be active during the entire procedure once the embolic protection system 110 is deployed, or it may be activated at intervals as needed or only at the end of the intervention. The gas capture system 150 can be activated as frequently as and for as long as the operator deems necessary to sufficiently remove gas bubbles.

It should be understood that elements of the embodiments can be combined, for example a closed-loop gas capture system may be integrated into the edges of the filters in second or third embodiments, or a detent may be integrated into the filters of the second or third embodiments. Likewise, a catheter terminating in a sealing member may be used with the first embodiment, such that the inner lumen terminates rather than forming a closed loop.

1. A vascular catheter including an embolic protection system which is deployable within a blood vessel, comprising:
a filter arranged to block the flow of solid particles; and,
a gas capture system for capturing gas bubbles entrained within a flow of blood along the blood vessel, the gas capture system comprising:
an outer lumen comprising one or more openings;
an inner lumen comprising one or more fluid collection ports; and,
a first source of pressure coupled to the inner lumen,
wherein the inner lumen and the outer lumen are controllable to move relative to each other to bring at least one of the one or more openings of the outer lumen into alignment with at least one of the one or more fluid collection ports, and wherein the first source of pressure is controllable to draw gas bubbles into the fluid collection ports.

2. The vascular catheter of claim 1, wherein the first source of pressure is one selected from a group comprising a first syringe connected to a first syringe controller, a pressure source and pressure regulator, and a pump and pump controller.

3. The vascular catheter of claim 1, wherein the first source of pressure is controllable to flush the inner lumen with a flushing fluid, preferably wherein the flushing fluid is one selected from a group comprising saline, glucose solution, dextrose solution, electrolyte solutions, fatty emulsions, and perfluorocarbons.

4. The vascular catheter of claim 1, further comprising a second source of pressure coupled to the inner lumen.

5. The vascular catheter of claim 4, wherein the second source of pressure is one selected from a group comprising a second syringe connected to a second syringe controller, a pressure source and pressure regulator, and a pump and pump controller.

6. The vascular catheter of claim 4, wherein the second source of pressure is controllable to flush the inner lumen with a flushing fluid, preferably wherein the flushing fluid is one selected from a group comprising saline, glucose solution, dextrose solution, electrolyte solutions, fatty emulsions, and perfluorocarbons.

7. The vascular catheter of claim 1, wherein the inner lumen is one selected from the group comprising a catheter, a microcatheter, a guide catheter, and a specialised guidewire.

8. The vascular catheter of claim 1, further comprising a sealing member at a distal end of the outer lumen, preferably wherein the sealing member is one selected from a group comprising a duckbill valve, an elastomeric self-sealing valve and a combination duckbill and elastomeric self-sealing valve.

9. The vascular catheter of claim 1, wherein the relative movement between the inner lumen and the outer lumen comprises translational movement and/or rotational movement.

10. The vascular catheter of claim 1, further comprising a control system positioned at a proximal end of the vascular catheter to control the relative movement of the inner and outer lumina.

11. A vascular catheter including an embolic protection system which is deployable within a blood vessel, comprising:
a filter arranged to block the flow of solid particles; and,
a gas capture system for capturing gas bubbles entrained within a flow of blood along the blood vessel, the gas capture system comprising:
an outer lumen comprising one or more openings; and,
a guidewire,
wherein in use, the guidewire is controllable to move relative to the outer lumen to occlude one or more of the openings, and wherein gas bubbles are drawn into the openings when the guidewire is retracted relative to the outer lumen.

12. The vascular catheter of claim 11, further comprising a sealing member at a distal end of the outer lumen, preferably wherein the sealing member is one selected from a group comprising a duckbill valve, an elastomeric self-sealing valve and a combination duckbill and elastomeric self-sealing valve.

13. The vascular catheter of claim 1, wherein the gas capture system is positioned at a location where gas bubbles accumulate.

14. The vascular catheter of claim 1, wherein the gas capture system is positioned at a leading edge of the filter.

15. The vascular catheter of claim 1, wherein the gas capture system is positioned at an apex of the filter.

16. The vascular catheter of claim 1, further comprising a bubble capture area, preferably wherein the bubble capture area is a detent.

17. The vascular catheter of claim 1, wherein the filter is arranged to trap solids.

18. The vascular catheter of claim 1, wherein the filter is arranged to deflect solids.

19. A method of providing embolic protection during a vascular procedure, comprising positioning a catheter over a guidewire within a blood vessel and controlling a gas capture system to capture gas bubbles introduced during the procedure and entrained within a flow of blood along the blood vessel.

20. The method of claim 19, wherein controlling a gas capture system to capture gas bubbles comprises aspirating gas bubbles through one or more openings.
